(19) United States
(12) Patent Application Publication
(10) Pub. No.: US 2006/0264739 A1
(43) Pub. Date: Nov. 23, 2006

(54) Method and control apparatus for controlling medical imaging systems

(75) Inventors: Arie Ariav, Doar-Na Hof Ashkelon (IL); Vladimir Ravitch, Ashkelon (IL); David Nitsan, Azur (IL)

Correspondence Address: Martin D. Moynihan;PRTSI, Inc., P.O. Box 16446, Arlington, VA, 22215, US

(73) Assignee: Nexense Ltd., Yavne (IL)

(21) Appl. No.: 11/417,145

(22) Filed: May 4, 2006

(30) Foreign Application Priority Data
May 5, 2005 (IL) 168424

(51) Int. Cl. A61B 5/05

(52) U.S. Cl. 600/413

(57) ABSTRACT

A method and apparatus for controlling an imaging system while imaging a patient's body or part thereof, by sensing a cyclically-occurring physiological condition of the patient over a plurality of cycles, and producing a sensor output signal corresponding thereto; analyzing the sensor output signal over the plurality of cycles to identify at least one time point of each cycle in which the sensor output signal exhibits a minimum deviation in the plurality of cycles; and utilizing the at least one time point of minimum deviation for gating the imaging system.

### RELATED APPLICATION

This application includes subject matter, and claims the priority date of Israel Patent Application No. 168424, filed on May 5, 2005, the content of which is incorporated herein by reference.

### FIELD AND BACKGROUND OF THE PRESENT INVENTION

The present invention relates to a method and apparatus for controlling medical imaging systems, such as magnetic resonance imaging (MRI) systems, and computer tomography (CT) systems, as well as X-ray, ultrasound, and other types of medical imaging systems. The invention particularly relates to controlling the gating of such imaging systems in order to reduce motion-generated artifacts therein resulting from movement of the patient during imaging, and thereby to produce sharper and clearer images.

The quality of the images produced in a medical imaging system depends to a large extent on the technique used for gating or synchronizing the operation of the imaging system. The common gating technique in present use is based on the ECG (electrocardiogram) signal of the patient, and therefore medical imaging systems in common use today include an ECG port for inputting the ECG signal in order to gate the operation of the imaging system. However, since the ECG signal does not closely correlate with actual body movements during imaging of the patient's body or part thereof, the quality of the so-produced images is degraded by motion-generated artifacts. This is true with respect to body motions derived from both the respiratory activity and the cardiac activity of the patient.

Many techniques have been proposed using sensors for sensing such cardiac and respiratory body movements and to use the output of such sensors for gating or synchronizing the imaging system. For example, U.S. Pat. No. 4,945,916, discloses an optical sensor for this purpose; and more recent U.S. Pat. Nos. 6,721,386 and 6,771,999 disclose mechanical sensors for this purpose. U.S. Pat. No. 6,621,278, and International Application No. IL02/00983 published Jun. 12, 2003 as Publication No. WO03/048688, both assigned to the assignee of the present application, and the contents of which are expressly incorporated herein by reference, disclose mechanical-type sensors of extremely high sensitivity which are particularly useful for this purpose.

Such methods, however, generally use a predefined feature along the cardiac cycle, e.g., the onset of systole or of diastole, as reference points for gating the imaging system. Therefore they do not take into account Heart Rate Variability (HRV) when existing in the patient being imaged. When HRV exists, which is not an uncommon occurrence, the gating signal used for synchronizing the imaging system becomes un-correlated to actual body motions in the patient, such that body motions introduce significant artifacts which blur and degrade the images produced.

### OBJECTS AND BRIEF SUMMARY OF THE PRESENT INVENTION

An object of the present invention is to provide a method of controlling a medical imaging system having advantages in the above respects, in that the method takes into account Heart Rate Variability (HRV) when existing in a patient. Another object of the present invention is to provide a control apparatus for controlling an imaging system in order to reduce the motion-generated artifacts produced by the imaging system, particularly when HRV exists. A further object of the present invention is to provide a medical imaging system capable of producing high-quality images with reduced motion-generated artifacts particularly when HRV exists in the patient.

According to one aspect of the present invention, there is provided a method of controlling an imaging system while imaging a patient's body or part thereof, comprising the steps: sensing a cyclically-occurring physiological condition of the patient over a plurality of cycles, and producing a sensor output signal corresponding thereto; analyzing the sensor output signal over the plurality of cycles to identify at least one time point of each cycle in which the sensor output signal exhibits a minimum deviation in the plurality of cycles; and utilizing the at least one time point of minimum deviation for gating the imaging system.

In the preferred embodiment of the invention described below, the sensing step senses a cyclically-occurring physiological movement of a portion of the patient's body. Particularly good results are obtainable when the sensor is of the displacement-type or accelerometer-type sensor described in the above-cited U.S. Pat. No. 6,621,278 or International Patent Application of Publication No. WO2004/0726578, assigned to the assignee of the present application the (contents of which have been incorporated herein by reference).

In the described preferred embodiment, the cyclically-occurring physiological movement sensed is a movement in the cardiac and/or respiratory system of the patient's body. Preferably, one or more time points of minimum deviation are identified by superimposing each cycle of the sensor output signal over the preceding cycles in the plurality of cycles, and detecting one or more time points of the superimposed cycles exhibiting minimum deviation.

In the described preferred embodiment, the one or more time points of minimum deviation are also used for enabling the imaging system in addition to gating the imaging system. After each time point of minimum deviation has been detected and utilized for enabling and gating the imaging system, the sensor output signal is continued to be analyzed and used for disabling the imaging system whenever the deviation of the respective time point falls outside a pre-selected tolerance limit.

The analysis may involve initially pre-selecting a plurality of initial time points of a cycle and analyzing the sensor output signal over the plurality of cycles to identify the initial time point of each cycle exhibiting a minimum deviation over the plurality of cycles. Alternatively, the analysis may involve pre-selecting a time point in a cycle, and analyzing the sensor output signal over a plurality of cycles until the pre-selected time point exhibits a predetermined minimum deviation over the plurality of cycles.

The invention is particularly useful where the imaging system includes a gating port for receiving an ECG gating signal since such a system already includes the various controls and the algorithms involved for controlling the operation of the imaging system, during both the data acquisition phase and the picture reconstruction phase. When the invention is used in such an imaging system, the identified time point or points, in which the sensor output signal exhibits a minimum deviation over the plurality of monitored cycles, are used for synthesizing a virtual ECG signal to be applied to the gating port of the imaging system for gating the imaging system.

In order to produce a gating signal which is more closely correlated to the actual body motions, two or more time points may be identified in the sensor output signal exhibiting a minimum deviation over the plurality of cycles, and may be used for synthesizing the virtual or ECG signal to be applied to the gating port of the imaging system for gating the imaging system. Thus, one such time point of minimum deviation may be used for generating a first segment of the virtual ECG signal, and one or more other time points of minimum deviation may be used for generating one or more other segments of the virtual ECG signal. Since the accumulated error in such a synthesized virtual ECG signal increases with time from the respective time point of minimum deviation, identifying two or more time points of minimum deviation, and using them for synthesizing the virtual ECG signal, reduces such an accumulated error.

According to another aspect of the present invention, there is provided control apparatus for an imaging system to be used for imaging a patient's body or part thereof, the control apparatus comprising: a sensor for sensing a cyclically-occurring physiological condition of the patient over a plurality of cycles, and for producing a sensor output signal corresponding thereto; and a processor designed to analyze the sensor output signal over the plurality of cycles to identify at least one time point of each cycle in which the sensor output signal exhibits a minimum deviation over the plurality of cycles, and to use the at least one time point of minimum deviation for gating the imaging system.

According to a further aspect of the present invention, there is provided an imaging system for imaging a patient's body, comprising: an imaging device for imaging the patient's body, and including a gating control for gating said imaging device in order to reduce motion-generated artifacts; a sensor for sensing a cyclically-occurring physiological condition of the patient over a plurality of cycles, and for producing a sensor output signal corresponding thereto; and a processor designed to analyze said sensor output signal over said plurality of cycles to identify at least one time point of each cycle in which the sensor output signal exhibits a minimum deviation in said plurality of cycles, and to use at least one time point of minimum deviation for gating said imaging device.

Further features and advantages of the invention will be apparent from the description below.

### BRIEF DESCRIPTION OF THE DRAWINGS

The invention is herein described, by way of example, with reference to the accompanying drawings, wherein:

FIG. 1 diagrammatically illustrates one form of control apparatus constructed in accordance with the present invention for controlling a medical imaging system;

FIG. 2 is a flowchart illustrating the main operations performed by the processor in the system of FIG. 1;

FIG. 3 illustrates a mechano-cardiogram (MCG) signal produced by the sensor in the apparatus of FIG. 1 correlated to the electrocardiogram (ECG) signal normally used for gating medical imaging systems;

FIGS. 4a-4c illustrate examples of how two time points of minimum deviation in the MCG (mechano-cardiogram) signal may be selected for gating the imaging system, and the relationship of the selected time points to the ECG signal;

FIG. 5 illustrates a typical MCG signal produced when an accelerometer-type sensor is used in the system of FIG. 1;

FIG. 6 is a magnified view of a portion of the signal of FIG. 5;

FIG. 7 illustrates a template that may be used for analyzing the signal of FIG. 6;

FIG. 8 illustrates the MCG output of the sensor in the system of FIG. 1 indicating the time point of closure of the aortic valve, and

FIGS. 9-11 illustrate waveforms showing how the processor identifies the time point or points of minimum deviation in the MCG signal outputted by the sensor, which time point or points are to be used in synthesizing the virtual ECG signal for gating the imaging system.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

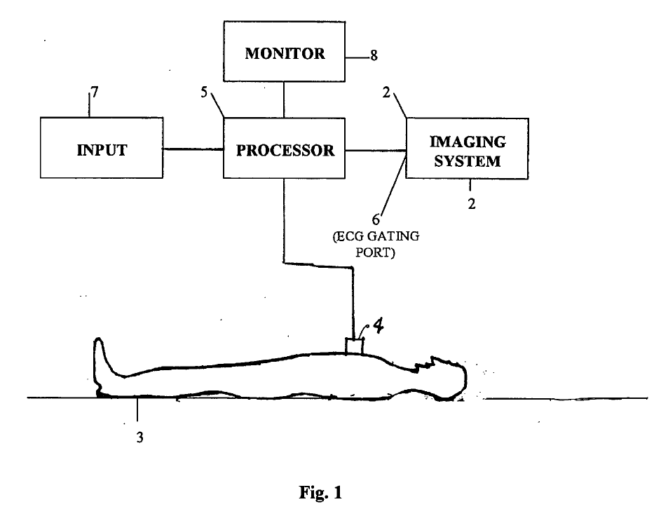

With reference first to FIG. 1, there is illustrated an imaging system, generally designated 2, for imaging the body, or a part of the body, of a patient 3 in accordance with the present invention. The imaging system illustrates a sensor 4 directly applied to the body or part thereof to be imaged, for sensing a cyclically-occurring physiological condition of the patient. In the described preferred embodiment, sensor 4 senses a physiologically-occurring physiological movement of the respective portion of the patient's body, such as a movement in the cardiac system of the patient's body, and/or a movement in the respiratory system of the patient's body. Preferably, sensor 4 is an accelerometer-type sensor, or a displacement-type sensor, as described in the above-cited U.S. Pat. No. 6,621,278, or International Patent Application No. WO03/048688, both assigned to the same assignee as the present application, and the contents of which have been incorporated herein by reference.

In accordance with the present invention, the imaging system includes a processor, generally designated 5, which analyzes the output signal from sensor 4 over a plurality of cycles to identify one or more time points of each cycle in which the sensor output signal exhibits a minimum deviation in the plurality of cycles. The processor utilizes the so-identified one or more time points of each cycle of minimum deviation for gating the imaging system 2.

In the described preferred embodiment, imaging system 2 may be an existing MRI or CT imaging system designed to utilize the ECG signal of the patient for gating and synchronizing the operation of the imaging system. Thus, the imaging system 2 itself includes the appropriate algorithms and controls for controlling the operation of the imaging system both in the data acquisition phase, and also in the picture reconstruction phase. Such imaging systems therefore commonly include an ECG gating port, shown at 6 in FIG. 1, for introducing the ECG gating signal. In the preferred embodiment of the invention illustrated in FIG. 1, the processor 5, which identifies one or more time points over a plurality of cycles in which the output signal from sensor 4 exhibits a minimum deviation, uses these time points of minimum deviation for synthesizing a “virtual ECG” signal. The latter signal is applied to the ECG gating port 6 of the imaging system, and thereby controls the operation of the imaging system in the same manner as would the conventional ECG signal in the respective imaging system.

The apparatus illustrated in FIG. 1 further includes an input device 7 which may be used to input information into processor 5, and also a monitor 8 enabling the user to visually monitor certain operations performed by processor 5, as will be described more particularly below.

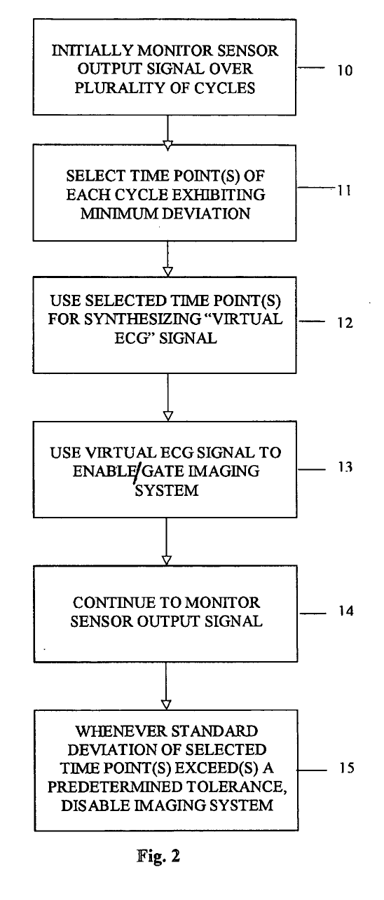

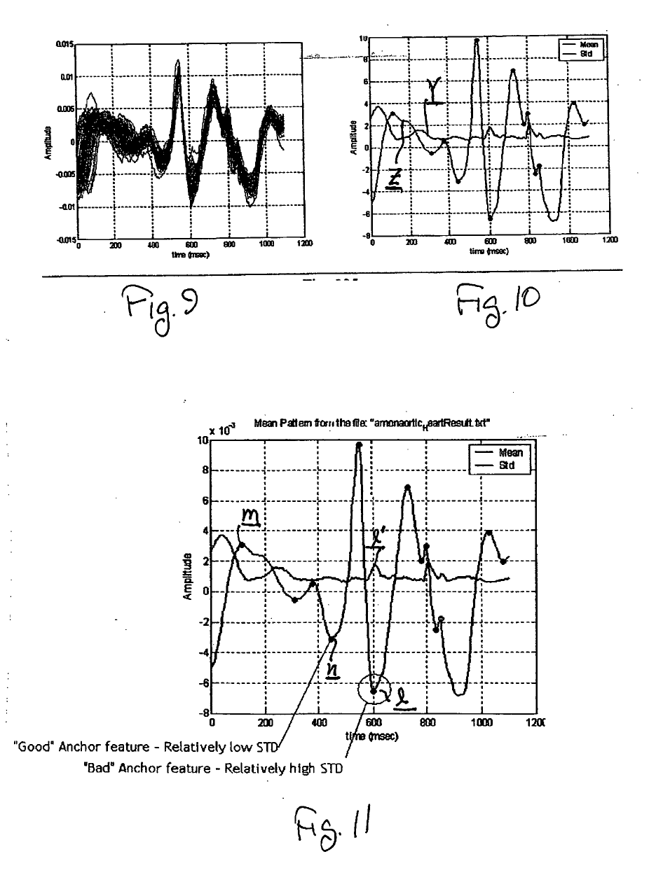

FIG. 2 is a flowchart broadly illustrating the overall operation of processor 5. As shown in FIG. 2, before the imaging system 2 is enabled, processor 5 initially monitors the output signal from sensor 4 over a plurality of cycles (e.g. 10-30 cycles), and selects one or more time points of each cycle exhibiting minimum deviation (block 11). As will be described below particularly with respect to FIG. 9, this is done by superimposing each cycle of the sensor output signal over the preceding cycles, and detecting the one or more time points of the superimposed cycles exhibiting minimum standard deviation.

As further shown in FIG. 2, processor 5 utilizes the selected one or more time points to synthesize a virtual ECG signal (block 12), which is applied to the ECG gating port 6 of the imaging system 2 to enable and gate the imaging system (block 13). By using such a virtual ECG signal for enabling and gating the imaging system, instead of the actual ECG signal derived from the patient, motion-generated artifacts, normally produced by the imaging system when gated by the actual ECG signal derived from the patient, are substantially reduced or eliminated even where Hard Rate Variability (HRV) exists in the patient.

While the synthesized ECG signal outputted from processor 5 is used for gating the imaging system 2, processor 5 continues to analyze the output signal of sensor 4 (block 14); and whenever it is found that the deviation of the selected time points exceeds a predetermined tolerance, the imaging system is disabled (block 15). Any data generated while the imaging system is disabled will therefore not be included in the data acquired by the imaging system during the data acquisition phase, or used for reconstructing the pictures in the picture reconstruction phase.

The basic flow chart illustrated in FIG. 2 may be varied in a number of respects, according to the particular application.

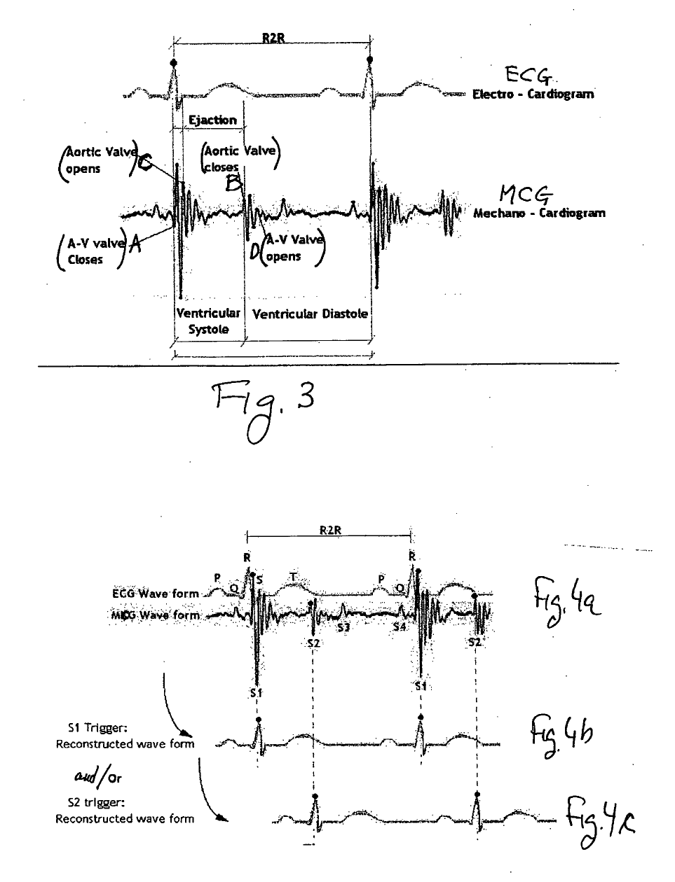

For example, it may be desirable to limit the time point of each cycle to be used as the gating signal for gating the imaging system to one (or more) of a plurality of pre-selected time points. FIG. 3 illustrates a number of pre-selected time points in a cardiac cycle which may be used for this purpose. These pre-selected time points would be analyzed over a plurality of cycles in order to identify the specific time point (or points) exhibiting a minimum deviation in the plurality of cycles monitored, and the so identified time point would be used for gating the imaging system.

For example, the upper signal illustrated in FIG. 3 represents the MCG (mechano-cardiogram) signal outputted from sensor 4, and includes a number of time points that may be pre-selected for possible use in gating the imaging system; whereas the lower signal in FIG. 3 illustrates the relationship of those pre-selected time points to the ECG (electrocardiogram) signal presently used for gating the imaging system. Thus, as shown in FIG. 3, the ventricular systole phase of the cardiac cycle, during which the heart muscle contracts to cause the forceful ejection of blood into the arterial system, normally extends from the point of closure of the A-V valve (point A, FIG. 3) and the time point of closure of the aortic valve (B, FIG. 3). Either of these time points may be pre-selected for possible use in gating the imaging system. Alternatively, it may be desirable to pre-select one or more different time points in the cardiac cycle for possible use in gating the imaging system, such as point C in FIG. 3, when the aortic valve opens, or point D in FIG. 3 when the A-V valve opens. In such case, only the pre-selected time points C and D in the MCG signal outputted from the sensor 4 would be analyzed by processor 5 to determine the time point (or points) of minimum deviation over the plurality of cycles monitored by the processor. The so-identified time points or points would be used in synthesizing the virtual ECG signal applied to port 6 of the imaging system 2 for gating the imaging system.

Instead of monitoring a plurality of pre-selected time points in a cardiac cycle, to identify the time point of minimum deviation for use in synthesizing the virtual ECG signal to be used for gating the imaging system, a single time point may be pre-selected and monitored for a number of cycles. In this case, such a pre-selected time point would be used for gating the imaging system only when the pre-selected time point deviation is found to be within a predefined limit.

The foregoing will be better understood by reference to FIGS. 4a-4c.

The upper waveform illustrated in FIG. 4a is a typical ECG signal derived from the patient; whereas the lower waveform in FIG. 4a is a typical MCG signal derived from sensor 4 applied to the patient. The MCG signal illustrates four possible time points (S1-S4) in a cardiac cycle that may be pre-selected for possible use in reconstructing the virtual ECG signal to be used for gating the imaging system. The relation of the time points S1-S4 in the MGC signal to the ECG signal is shown by the upper ECG waveform of FIG. 4a. FIG. 4b illustrates the virtual ECG signal synthesized by processor 5 when time point S1 is selected for use as the gating signal when its deviation over a plurality of cycles falls within a predefined tolerance range; whereas FIG. 4c illustrates the synthesized virtual ECG signal when time point S2 is selected for use as the gating signal when its deviation over a plurality of cycles falls within a predefined range.

It will also be appreciated that two or more time points could be identified in the sensor output signal for analysis and for use as gating pulses when the deviation of the respective time point over a plurality of monitored cycles falls within a predefined low range. As will be described more particularly below particularly with respect to FIGS. 9-11, such a variation may be used in order to reduce the accumulated error. This is done by constructing the virtual ECG signal to be used for gating the imaging system of a plurality of segments of the cardiac cycles, each segment being defined by a time point exhibiting minimum deviation over the plurality of monitored cycles.

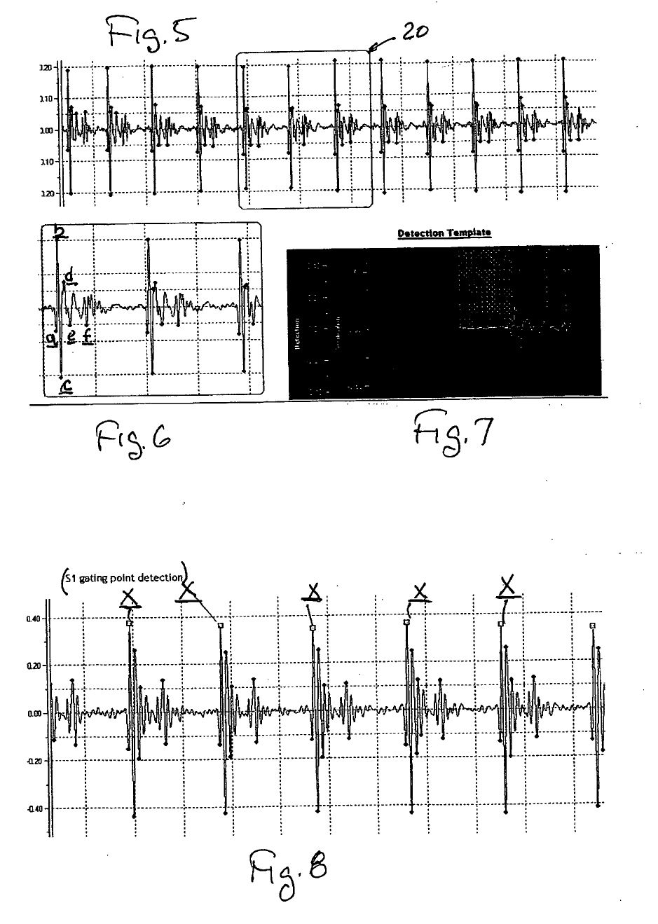

Monitor 8 in the apparatus of FIG. 1 displays the MCG signal outputted by sensor 4 and the processing of that signal by processor 5. FIG. 5 illustrates a typical MCG signal displayed in monitor 8 when sensor 4 is of the accelerometer-type, e.g., as described in the above-cited Publication No. WO03/048688. FIG. 6 is a magnified portion of the signal display in FIG. 5 within the window 20; and FIG. 7 illustrates a template used for analyzing the signal of FIG. 6. All the foregoing are displayed in monitor 8 during the processing of the MCG signal outputted from sensor 4. Points a-f illustrate possible time points which may be selected for monitoring and used in synchronizing the gating signal when the respective time point is found to deviate a minimum amount (i.e. within a predefined range) over a plurality of monitored cycles.

FIG. 8 illustrates a typical MCG signal outputted by sensor 4 when the sensor is of the acceleromometer type. Point “X” in FIG. 8 illustrates the time point in each of the cardiac cycles indicating the closure of the aortic valve. Such time point may be used alone, or with other time points, in synthesizing the virtual ECG signal to be used for gating the imaging system when the deviation of the respective time point falls within a predetermined limited range during a plurality of monitored cycles.

FIG. 9 illustrates the manner in which the aortic time point “X” in the signal of FIG. 8 is analyzed over a plurality of cycles in order to determine its standard deviation, or deviation. FIG. 10 illustrates, in waveform Y the standard deviation of the aortic time point X during a plurality of monitored cycles, and at point Z the mean deviation over the plurality of cycles. FIG. 11 is a magnified view of FIG. 10.

As shown in FIG. 9, a plurality of the cardiac cycles are superimposed on each other to produce a composite waveform varying in thickness at each time point corresponding to the deviation exhibited by the respective time point during a plurality of monitored cycles. Thus, a point on the composite waveform of minimum thickness will indicate a time point of minimum deviation. In FIGS. 10 and 11, waveform Y represents the deviation of a particular time point, in this case the deviation of the monitored aortic time point X of FIG. 8, during the monitored cycles. Waveform Y in FIGS. 10 and 11 illustrates the mean deviation of the superimposed cycles.

It will be seen that, in FIGS. 10 and 11, time point “l” represents a point of the cardiac cycle involving a large standard deviation, because of the “blip” l in the standard deviation curve Y at that point, and therefore this time point would not be a good time point for gating the imaging system. Time point “m” of the mean deviation curve Z would also not be a good time point for gating the imaging system because this time point is in the region of the standard deviation curve Y which exhibits a large variation over the plurality of monitored cycles. On the other hand, time point “n” in the mean deviation curve Z is in the region wherein the standard deviation of the selected time point “X” is in a region of the standard deviation curve Y which is relatively low and steady. Time point “n” would therefore be a good time point for gating the imaging system.

Accordingly, processor 5 (FIG. 1), after analyzing the MCG signal outputted from sensor 4 over a plurality of cardiac cycles, would identify time point “n” of FIGS. 10 and 11 as a suitable time point of minimum deviation for use in gating the imaging system 2, and would therefore synthesize a virtual ECG signal, based on time point “n”, to be applied to the ECG port 6 of the imaging system.

As indicated earlier, using a single time point for synthesizing the gating signal produces an accumulative error, namely an error which increases with time from the respective time point. In order to reduce this accumulative error, two or more time points may be determined each representing a minimum deviation of a part or segment of the cardiac cycle, and the two or more such determined time points may be used for synthesizing the virtual ECG wave used for gating the imaging system. For example, in the segment of the cardiac cycle represented by time point “m”, in a region in which there is a wide variation in the standard deviation as shown by curve “Y”, a point of minimum deviation may be selected in this segment of the cardiac cycle and used for synthesizing this segment of the virtual ECG wave used for gating the imaging system, such that the gating wave will include gating pulses at two (or more) time points of the cardiac cycle.

While the invention has been described with respect to one preferred embodiment, it will be appreciated that may variations and other applications of the invention may be made. For example, the sensor 4 could sense cyclically-occurring movements in the respiratory system, rather than in the cardiac system, of the patient's body for gating the imaging system Also, the imaging system could be an MRI system, a CT system, or one of the other known imaging systems. Where the imaging system is one exposing the patient to potentially harmful radiation, such as a CT system, the gating pulses could be used not only for enabling the imaging system with respect to the data-acquisition phase and the picture-reconstruction phase, but also for activating the radiation production in the system, so as to minimize the patient's exposure to such radiation. In addition, the monitored periods for determining time points of minimum deviation over a plurality of cycles could be a predetermined number of cycles (e.g. 10-30 cycles), or could be indefinite periods, extending until the monitored time point exhibits the desired minimum deviation, or exceeds a specified maximum deviation.

Many other variations, modifications and applications of the invention will be apparent.

What is claimed is:

1. A method of controlling an imaging system while imaging a patient's body or part thereof, comprising the steps:
sensing a cyclically-occurring physiological condition of the patient over a plurality of cycles, and producing a sensor output signal corresponding thereto;
analyzing said sensor output signal over said plurality of cycles to identify at least one time point of each cycle in which the sensor output signal exhibits a minimum deviation in said plurality of cycles;
and utilizing said at least one time point of minimum deviation for gating said imaging system.

2. The method according to claim 1, wherein said sensing step senses a cyclically-occurring physiological movement of a portion of the patient's body.

3. The method according to claim 2, wherein said cyclically-occurring physiological movement is a movement in the cardiac system of the patient's body.

4. The method according to claim 2, wherein said cyclically-occurring physiological movement is a movement in the respiratory system of the patient's body.

5. The method according to claim 1, wherein at least one time point of minimum deviation is identified by superimposing each cycle of the sensor output signal over the preceding cycles in said plurality of cycles, and detecting the at least one time point of the superimposed cycles exhibiting minimum deviation.

6. The method according to claim 1, wherein said at least one time point of minimum deviation is used for enabling, as well as for gating, said imaging system.

7. The method according to claim 6, wherein after said at least one time point of minimum deviation has been detected and utilized for enabling and gating said imaging system, said sensor output signal is continued to be analyzed and used for disabling the imaging system whenever the deviation of the at least one time point falls outside a pre-selected tolerance limit.

8. The method according to claim 1, wherein said analysis step involves initially pre-selecting a plurality of initial time points of a cycle, and analyzing said sensor output signal over said plurality of cycles to identify the initially-pre-selected time point of each cycle exhibiting a minimum deviation over said plurality of cycles.

9. The method according to claim 1, wherein said analysis step involves pre-selecting a time point in a cycle, and analyzing said sensor output signal over a plurality of cycles until said pre-selected time point exhibits a predetermined minimum deviation over said plurality of cycles.

10. The method according to claim 1, wherein said imaging system includes a gating port for receiving an ECG gating signal, and wherein said identified at least one time point, in which the sensor output signal exhibits a minimum deviation over said plurality of cycles, is used for synthesizing a virtual ECG signal to be applied to said gating port of the imaging system for gating the imaging system.

11. The method according to claim 9, wherein two or more time points are identified in the sensor output signal in which the sensor output signal exhibits a minimum deviation over said plurality of cycles, and are used for synthesizing the virtual ECG signal to be applied to said gating port of the imaging system for gating the imaging system.

12. Control apparatus for controlling an imaging system to be used for imaging a patient's body or part thereof, said control apparatus comprising:
a sensor for sensing a cyclically-occurring physiological condition of the patient over a plurality of cycles, and for producing a sensor output signal corresponding thereto;
and a processor designed to analyze said sensor output signal over said plurality of cycles to identify at least one time point of each cycle in which the sensor output signal exhibits a minimum deviation in said plurality of cycles, and to use said at least one time point of minimum deviation for gating said imaging system.

13. The control apparatus according to claim 12, wherein the processor identifies said at least one time point of minimum deviation by superimposing each cycle of the sensor output signal over the preceding cycles in said plurality of cycles, and detecting the at least one time point of the superimposed cycles exhibiting minimum deviation.

14. The control system according to claim 12, wherein said processor utilizes said at least one time point of minimum deviation for enabling, as well as for gating, said imaging system.

15. The control system according to claim 14, wherein said processor, after said at least one time point of minimum deviation has been detected and utilized for enabling and gating said imaging system, continues to analyze said sensor output signal and to use same for disabling the imaging system whenever the deviation of the at least one time point falls outside a pre-selected tolerance limit.

16. The control system according to claim 12, wherein said processor, in said analysis step, initially pre-selects a plurality of time points of a cycle, and analyzes said sensor output signal over said plurality of cycles to identify the pre-selected time point of each cycle exhibiting a minimum deviation over said plurality of cycles.

17. The control system according to claim 12, wherein said processor analyzes said sensor output signal over a plurality of cycles until a pre-selected time point exhibits a predetermined minimum deviation over said plurality of cycles.

18. The control system according to claim 12, wherein said imaging system includes a gating port for receiving an ECG gating signal; and wherein said processor utilizes said identified at least one time point, in which the sensor output signal exhibits a minimum deviation in said plurality of cycles, for synthesizing a virtual ECG signal to be applied to said gating port of the imaging system for gating the imaging system.

19. The control system according to claim 18, wherein said processor identifies two or more time points in the sensor output signal in which the sensor output signal exhibits minimum deviation over said plurality of cycles, and uses said two or more identified time points for synthesizing the virtual ECG signal to be applied to said gating port of the imaging system for gating the imaging system.

20. An imaging system for imaging a patient's body comprising:
an imaging device for imaging the patient's body, and including a gating control for gating said imaging device in order to reduce motion-generated artifacts;
a sensor for sensing a cyclically-occurring physiological condition of the patient over a plurality of cycles, and for producing a sensor output signal corresponding thereto;
and a processor designed to analyze said sensor output signal over said plurality of cycles to identify at least one time point of each cycle in which the sensor output signal exhibits a minimum deviation in said plurality of cycles, and to use at least one time point of minimum deviation for gating said imaging device.

21. The imaging system according to claim 20, wherein said sensor senses movement in the cardiac system of the patient's body.

22. The imaging system according to claim 20, wherein said senses movement in the respiratory system of the patient's body.

23. The imaging system according to claim 20, wherein the processor identifies said at least one time point of minimum deviation by superimposing each cycle of the sensor output signal over the preceding cycles in said plurality of cycles, and detecting the at least one time point of the superimposed cycles exhibiting minimum deviation.

24. The imaging system according to claim 20, wherein said processor utilizes said at least one time point of minimum deviation for enabling, as well as for gating, said imaging system.

25. The imaging system according to claim 24, wherein said processor, after said at least one time point of minimum deviation has been detected and utilized for enabling and gating said imaging system, continues to analyze said sensor output signal and to use same for disabling the imaging system whenever the deviation of the at least one time point falls outside a pre-selected tolerance limit.

26. The imaging system according to claim 24, wherein said processor, in said analysis step, initially pre-selects a plurality of time points of a cycle, and analyzes said sensor output signal over said plurality of cycles to identify the pre-selected time point of each cycle exhibiting a minimum deviation over said plurality of cycles.

27. The imaging system according to claim 20, wherein said processor analyzes said sensor output signal over a plurality of cycles until a pre-selected time point exhibits a predetermined minimum deviation over said plurality of cycles.

28. The imaging system according to claim 20, wherein said imaging device includes a gating port for receiving an ECG gating signal; and wherein said processor utilizes said identified at least one time point, in which the sensor output signal exhibits a minimum deviation in said plurality of cycles, for synthesizing a virtual ECG signal to be applied to said gating port of the imaging device for gating the imaging system.

29. The imaging system according to claim 20, wherein said processor identifies two or more time points in the sensor output signal in which the sensor output signal exhibits minimum deviation over said plurality of cycles, and uses said two or more identified time points for synthesizing the virtual ECG signal to be applied to said gating port of the imaging device for gating the imaging system.
